(19) United States
(12) Patent Application Publication
(10) Pub. No.: US 2022/0274944 A1
(43) Pub. Date: Sep. 1, 2022

(54) COMPOSITE ABSORBENT AND USE THEREOF

(71) Applicant: Institute of Process Engineering, Chinese Academy of Sciences, Beijing (CN)

(72) Inventors: Weiguo Cheng, Beijing (CN); Li Dong, Beijing (CN); Junjie Chu, Beijing (CN); Zengliang Zhang, Beijing (CN); Qian Su, Beijing (CN); Songsong Chen, Beijing (CN); Yifan Liu, Beijing (CN); Xiangping Zhang, Beijing (CN); Suojiang ZHANG, Beijing (CN)

(73) Assignee: Institute of Process Engineering, Chinese Academy of Sciences, Beijing (CN)

(21) Appl. No.: 17/637,241

(22) PCT Filed: Mar. 23, 2020
(86) PCT No.: PCT/CN2020/080560

§ 371 Date: Feb. 22, 2022

(30) Foreign Application Priority Data
Aug. 23, 2019 (CN) 201910785196.2
Aug. 23, 2019 (CN) 201910786256.2

(51) Int. Cl. C07D 317/38; C07D 301/32; B01D 53/14

(52) U.S. Cl. CPC C07D 317/38; C07D 301/32; B01D 53/14

(57) ABSTRACT

Disclosed are a composite absorbent and a method for using same in the absorption and conversion of ethylene oxide for the coupling co-production of ethylene carbonate. The composite absorbent comprises an ionic liquid and ethylene carbonate, wherein the ionic liquid is an imidazole ionic liquid, a quaternary ammonium ionic liquid and a quaternary phosphonium ionic liquid. The composite absorbent is used for absorbing ethylene oxide and carbon dioxide, and is also used in the absorption and conversion of ethylene oxide for the coupling co-production of ethylene carbonate.

### TECHNICAL FIELD

The present application belongs to the technical field of chemical engineering, for example, relates to a composite absorbent and a method thereof for coupled absorption and conversion of ethylene oxide and co-production of ethylene carbonate and to a composite absorbent and a method thereof for separation and purification of ethylene oxide.

### BACKGROUND

Ethylene carbonate, known as a green chemical raw material in the 21st century, is widely used as a chemical intermediate and a good solvent. Ethylene oxide, one of raw materials for the synthesis of ethylene carbonate, is mainly generated by oxidizing ethylene under the action of a silver-containing catalyst. In the process of preparing ethylene oxide, impurity gases such as methane and ethane are coexisting. To obtain refined ethylene oxide, in the industry, ethylene oxide is typically absorbed from a gas mixture by an absorption method and then purified through steam stripping, concentration and another processes to obtain high purity ethylene oxide. The refined ethylene oxide and carbon dioxide are catalyzed in a reaction kettle so that ethylene carbonate is synthesized.

Meanwhile, ethylene oxide (EO) is also an important petrochemical product with good sterilization and disinfection effects and mainly used in washing, pharmaceutical and printing and dyeing industries. At present, ethylene oxide is mainly produced by an ethylene oxidation method. Ethylene is reacted with oxygen in the presence of a silver catalyst to produce ethylene oxide. However, due to a low conversion rate and low selectivity, ethylene oxide is generated as well as some gases such as methane, ethane and carbon dioxide, and unreacted ethylene also exists. To prepare relatively pure ethylene oxide in the industry, generally, with water as an absorbent, the relatively pure ethylene oxide is obtained through desorption, concentration and refinement.

At present, a process in which water is used as the absorbent has problems such as a complicated flow, poor operation elasticity and high energy consumption. Many domestic and foreign patents have made improvements as for this point. U.S. Pat. No. 3,948,621A1 discloses the use of methanol as an absorbent for separation and purification of ethylene oxide. This absorbent has a good absorption effect and low energy consumption during desorption. However, methanol easily reacts with ethylene oxide so that the absorption temperature needs to be strictly controlled. Moreover, methanol is easily lost with the volatilization of desorption gases during desorption. U.S. Pat. No. 4,221,727A1 discloses the use of ethylene carbonate as an absorbent for absorption and separation of ethylene oxide. With ethylene carbonate as the absorbent, not only a good absorption effect is achieved but also a side reaction can be avoided in an absorption process. However, in a desorption process, at a desorption temperature of 150° C. and under a pressure of 5 kPa, part of ethylene carbonate will volatilize with desorption gases, resulting in a loss of a solvent. Similarly, U.S. Pat. No. 5,559,255A discloses the use of propylene carbonate as an absorbent and CN102911137A discloses the use of a mixture of ethylene carbonate and water as an absorbent for ethylene oxide. However, neither of them solves the problem of a volatilization loss of the solvent during steam stripping. CN109422708A discloses a method that uses triethylene glycol dimethyl ether and an ionic liquid as absorbents. Such absorbents increase the absorbency and selectivity of ethylene oxide. However, the steam stripping of ethylene oxide still consumes a lot of energy during the whole process flow, and the process flow is complex, the equipment is at a high cost, and a one-time investment is large.

Therefore, to better absorb ethylene oxide in the gas mixture after ethylene oxidation and produce ethylene carbonate, a new absorbent and process are still needed, which can not only achieve highly efficient absorption of ethylene oxide but also solve the problem of high energy consumption during steam stripping.

### SUMMARY

An object of the present application is to provide a composite absorbent and a method thereof for coupling absorption with conversion of ethylene oxide and co-production of ethylene carbonate. In the present application, the composite absorbent of an ionic liquid and ethylene carbonate is used for absorbing ethylene oxide and carbon dioxide. In an absorption process, the ionic liquid may also be used as a catalyst to make the absorbed ethylene oxide and carbon dioxide react into ethylene carbonate, which improves an absorption effect of ethylene oxide, reduces desorption and refinement of ethylene oxide, and reduces energy consumption through simultaneous pre-conversion and absorption, thus having a good industrial application value.

Another object of the present application is to provide a composite absorbent and a method thereof for separation and purification of ethylene oxide. The absorbent includes an ionic liquid and ethylene carbonate, where the ionic liquid has a structure as represented by Formula I. The composite absorbent of the present application contains the ionic liquid having a particular structure. The use of the composite absorbent for the separation and purification of ethylene oxide can effectively improve an absorption rate of ethylene oxide and a cycle utilization rate of the composite absorbent, reducing a cost and energy consumption.

To achieve the objects, the present application adopts technical solutions described below.

In a first aspect, the present application provides a composite absorbent. The composite absorbent includes an ionic liquid and ethylene carbonate, where the ionic liquid has a structure represented by Formula I:

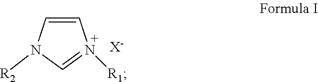

where R₁ and R₂ in Formula I are each independently selected from any one of substituted or unsubstituted C1-C6 alkyl, substituted or unsubstituted C2-C6 alkenyl, substituted or unsubstituted C2-C6 alkynyl, substituted or unsubstituted C6-C30 aryl, substituted or unsubstituted C3-C30 heteroaryl or substituted or unsubstituted C1-C6 alkoxy; and where an anion X⁻ in Formula I is selected from any one of BF₄⁻, PF₆⁻, Tf₂N⁻, RCOO⁻, Cl⁻ or Br⁻; where R is selected from any one of alkyl, alkenyl or alkynyl.

As used herein, the term “C1-C6 alkyl” refers to linear or branched alkyl having 1, 2, 3, 4, 5 or 6 carbon atoms and includes, without limitation, methyl, ethyl, n-propyl, isopropyl, n-butyl, isobutyl, sec-butyl, tert-butyl, n-pentyl, isopentyl, n-hexyl and the like. The term “C2-C6 alkenyl” refers to linear or branched alkenyl having 2, 3, 4, 5 or 6 carbon atoms and including at least one double bond in its molecular chain and includes, without limitation, —CH═CH(CH₃), —CH═C(CH₃)₂, —C(CH₃)═CH₂, —C(CH₃)═CH(CH₃), —C(CH₂CH₃)═CH₂, butadienyl, pentadienyl, hexadienyl or the like. The term “C2-C6 alkynyl” refers to linear or branched alkynyl having 2, 3, 4, 5 or 6 carbon atoms and includes, without limitation, —C≡CH, —C≡C(CH₃), —C≡C(CH₂CH₃), —CH₂C≡CH, —CH₂C≡C(CH₃), —CH₂C≡C(CH₂CH₃) or the like. The term “C6-C30 aryl” refers to aryl having 6-30 carbon atoms, which may contain, for example, 6, 12 or 18 carbon atoms, and includes, without limitation, phenyl, naphthyl, biphenyl or the like. The term “C3-C30 heteroaryl” refers to an aromatic ring system such as a monocyclic, bicyclic or tricyclic ring that has 3, 4, 5, 6, 7, 8, 9, 10, 11, 12, 13, 14, 15, 16, 17, 18, 19, 20, 21, 22, 23, 24, 25, 26, 27, 28, 29 or 30 ring atoms and contains at least one heteroatom which may be the same or different, such as oxygen, nitrogen or sulfur. Moreover, the “C3-C30 heteroaryl” may be benzo-fused in various cases and includes, without limitation, thienyl, furyl, pyrrolyl, oxazolyl, thiazolyl, imidazolyl, pyrazolyl, isoxazolyl, isothiazolyl, oxadiazolyl, triazolyl, benzofuryl, benzothienyl, benzoxazolyl, benzoisoxazolyl, benzimidazolyl, benzotriazolyl, indazolyl, indolyl, isoindolyl, pyridyl, pyridazinyl, pyrimidyl, pyrazinyl, triazinyl, quinolinyl, quinazolinyl, isoquinolinyl or the like. The term “C1-C6 alkoxy” refers to linear or branched alkoxy having 1, 2, 3, 4, 5 or 6 carbon atoms and includes, without limitation, methoxy, ethoxy, n-propoxy, isopropoxy, tert-butoxy or the like.

The composite absorbent containing the ionic liquid of the present disclosure has a relatively high absorbency for ethylene oxide and can significantly increase selectivity for absorption and separation of ethylene oxide, effectively reduce a vapor pressure of the absorbent, and reduce a loss of a solvent during desorption. The composite absorbent has the characteristics of a simple process flow, high operation elasticity, low energy consumption and a remarkable absorption effect and has a good industrial application prospect.

Optionally, the anion X⁻ is selected from any one of BF₄⁻, PF₆⁻, Tf₂N⁻ or RCOO⁻. Optionally, the anion X⁻ is BF₄⁻ or PF₆⁻.

Optionally, the substituents in R₁ and R₂ are each independently selected from any one of a hydroxyl group, an amino group, a nitro group, an aldehyde group, an ester group, a carboxyl group or a sulfhydryl group.

Optionally, the substituents in R₁ and R₂ are a hydroxyl group or an amino group.

As an optional technical solution of the present disclosure, the ionic liquid is selected from any one or a combination of at least two of 1-hydroxyethyl-3-methylimidazolium hexafluorophosphate, 1-hydroxyethyl-3-methylimidazolium tetrafluoroborate, 1-aminoethyl-3-methylimidazolium tetrafluoroborate, 1-aminoethyl-3-methylimidazolium hexafluorophosphate, 1-hydroxyethyl-3-ethylimidazolium hexafluorophosphate or 1-hydroxyethyl-3-ethylimidazolium tetrafluoroborate.

Optionally, a mass percentage of the ionic liquid in the composite absorbent is 10-60%, for example, may be 10%, 12%, 15%, 17%, 19%, 20%, 23%, 25%, 27%, 30%, 33%, 35%, 37%, 38%, 40%, 42%, 45%, 49%, 50%, 53%, 55%, 59%, 60% or the like. Optionally, the mass percentage is 30-50%.

In the present disclosure, preferably, a mass ratio of the ionic liquid to ethylene carbonate is controlled to be within the above range. When the content of the ionic liquid is too high, the composite absorbent has increased viscosity and cannot be in full contact with ethylene oxide, which might decrease absorption efficiency of ethylene oxide. When the content of the ionic liquid is too low, the selective absorbency of the composite absorbent decreases, which affects the absorption effect of ethylene oxide.

In a second aspect, the present disclosure further provides a method for separation and purification of ethylene oxide. The method uses the composite absorbent as described in the first aspect.

The method for separation and purification of ethylene oxide in the present disclosure uses the composite absorbent of an ionic liquid and ethylene carbonate, and can effectively improve the absorbency of ethylene oxide in a feed gas, simplify a process flow, increase operating elasticity of a device, and reduce energy consumption, which provides technical support for continuous production and purification of ethylene oxide.

Optionally, the method includes the following steps: making the composite absorbent in contact with a feed gas containing ethylene oxide, returning a lean gas mixture with ethylene oxide removed to an ethylene oxidation stage, and subjecting an ethylene oxide-rich absorption liquid to desorbing to obtain ethylene oxide.

Components of the feed gas in the present disclosure and molar percentages thereof are 2.6% ethylene oxide, 52.77% methane, 32.53% ethylene, 4.9% oxygen, 1.5% carbon dioxide, 4.18% argon, 1% nitrogen and 0.52% ethane, respectively. The feed gas used in the present disclosure includes a low content of ethylene oxide which is separated and purified by the composite absorbent of the ionic liquid and ethylene carbonate so that ethylene oxide with a relatively high purity can be obtained, and absorption efficiency is significantly improved.

Optionally, the method includes the following steps: making the composite absorbent in full countercurrent contact with the feed gas containing ethylene oxide in an absorption tower, returning the lean gas mixture with ethylene oxide removed at a top of the tower to the ethylene oxidation stage after the lean gas mixture is treated, feeding the ethylene oxide-rich absorption liquid at a bottom of the tower into a desorption tower after heat exchange, followed by collecting a desorbed gas phase at a top of the tower to obtain ethylene oxide, and returning a lean desorption liquid in a desorbed liquid phase at the bottom of the tower to the absorption tower after heat exchange.

Optionally, a molar concentration of ethylene oxide in the feed gas is 0.1-5%, for example, may be 0.1%, 0.3%, 0.5%, 0.8%, 0.9%, 1.0%, 1.2%, 1.5%, 2%, 2.5%, 2.6%, 2.8%, 2.8%, 3.0%, 3.5%, 4.0%, 4.5%, 5% or the like. Optionally, the molar concentration is 2-3%.

Optionally, an inlet temperature of the feed gas is 40-100° C., for example, may be, 40° C., 42° C., 43° C., 45° C., 48° C., 50° C., 51° C., 56° C., 59° C., 60° C., 65° C., 68° C., 70° C., 72° C., 73° C., 75° C., 78° C., 79° C., 80° C., 83° C., 85° C., 87° C., 90° C., 92° C., 95° C., 97° C., 99° C., 100° C. or the like. Optionally, the inlet temperature is 50-80° C.

Optionally, an operation pressure of the absorption tower is 0.1-5 MPa, for example, may be 0.1 MPa, 0.2 MPa, 0.3 MPa, 0.5 MPa, 0.8 MPa, 1.0 MPa, 1.2 MPa, 1.5 MPa, 1.9 MPa, 2.0 MPa, 2.3 MPa, 2.5 MPa, 2.6 MPa, 2.9 MPa, 3.0 MPa, 3.5 MPa, 3.8 MPa, 4.0 MPa, 4.2 MPa, 4.5 MPa, 5.0 MPa or the like. Optionally, the operation pressure is 1-3 MPa.

Optionally, an operation temperature of the absorption tower is 40-100° C., for example, may be 40° C., 42° C., 43° C., 45° C., 48° C., 50° C., 51° C., 56° C., 59° C., 60° C., 65° C., 68° C., 70° C., 72° C., 73° C., 75° C., 78° C., 79° C., 80° C., 83° C., 85° C., 87° C., 90° C., 92° C., 95° C., 97° C., 99° C., 100° C. or the like. Optionally, the operation temperature is 50-80° C.

Optionally, a molar ratio of the composite absorbent to the feed gas is (1-4):1, for example, may 15 be 1:1, 1.5:1, 1.7:1, 2:1, 2.1:1, 2.3:1, 2.5:1, 2.6:1, 2.7:1, 2.8:1, 2.9:1, 3:1, 3.2:1, 3.5:1, 3.6:1, 3.9:1, 4:1 or the like. Optionally, the molar ratio is (2-3):1.

Optionally, an operation pressure of the desorption tower is 10-150 kPa, for example, may be 10 kPa, 12 kPa, 15 kPa, 18 kPa, 20 kPa, 25 kPa, 30 kPa, 36 kPa, 38 kPa, 40 kPa, 45 kPa, 48 kPa, 50 kPa, 52 kPa, 55 kPa, 60 kPa, 62 kPa, 65 kPa, 70 kPa, 75 kPa, 80 kPa, 85 kPa, 90 kPa, 94 kPa, 100 kPa, 110 kPa, 120 kPa, 130 kPa, 135 kPa, 140 kPa, 145 kPa, 150 kPa or the like. Optionally, the operation pressure is 50-150 kPa.

Optionally, an operation temperature of the desorption tower is 80-150° C., for example, may be 80° C., 84° C., 85° C., 87° C., 89° C., 90° C., 95° C., 98° C., 100° C., 105° C., 108° C., 110° C., 115° C., 120° C., 125° C., 130° C., 134° C., 140° C., 145° C., 148° C., 150° C., or the like. Optionally, the operation temperature is 90-130° C.

Optionally, the lean desorption liquid at the bottom of the desorption tower is recycled back to the absorption tower after heat exchange to 50-80° C., which, for example, may be 50° C., 53° C., 54° C., 55° C., 57° C., 60° C., 63° C., 65° C., 68° C., 70° C., 72° C., 73° C., 75° C., 78° C., 79° C., 80° C. or the like.

Optionally, the ethylene oxide-rich absorption liquid at the bottom of the absorption tower is fed into the desorption tower after heat exchange to 90-130° C., which, for example, may be 90° C., 92° C., 93° C., 95° C., 98° C., 99° C., 100° C., 102° C., 105° C., 108° C., 110° C., 115° C., 118° C., 120° C., 124° C., 125° C., 126° C., 128° C., 129° C., 130° C. or the like.

In a third aspect, the present disclosure provides a composite absorbent. The composite absorbent includes an ionic liquid and ethylene carbonate, where the ionic liquid has a structure represented by Formula II or Formula III:

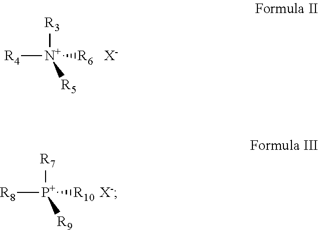

where R₃, R₄, R₅ and R₆ in Formula II are each independently selected from any one of substituted or unsubstituted C1-C6 alkyl, substituted or unsubstituted C2-C6 alkenyl, substituted or unsubstituted C2-C6 alkynyl, substituted or unsubstituted C6-C30 aryl, substituted or unsubstituted C3-C30 heteroaryl or substituted or unsubstituted C1-C6 alkoxy; where R₇, R₈, R₉ and R₁₀ in Formula III are each independently selected from any one of substituted or unsubstituted C1-C6 alkyl, substituted or unsubstituted C2-C6 alkenyl, substituted or unsubstituted C2-C6 alkynyl, substituted or unsubstituted C6-C30 aryl, substituted or unsubstituted C3-C30 heteroaryl or substituted or unsubstituted C1-C6 alkoxy; and where anions X⁻ in Formula II and Formula III are each independently selected from any one of Cl⁻, Br⁻ or I⁻.

In the present application, the composite absorbent of the ionic liquid and ethylene carbonate is used for absorbing ethylene oxide and carbon dioxide. In an absorption process, the ionic liquid may also be used as a catalyst to make the absorbed ethylene oxide and carbon dioxide react into ethylene carbonate, which improves an absorption effect of ethylene oxide, reduces desorption and refinement of ethylene oxide, and reduces energy consumption through simultaneous pre-conversion and absorption, thus having a good industrial application value.

In the present application, the anion in the ionic liquid in the composite absorbent is selected from any one of particular Cl⁻, Br⁻ or I⁻. Cations in the ionic liquid can cause polarization of a C—O bond of ethylene oxide, and at the same time a halogen ion attacks β-carbon atom with relatively small steric hindrance in an epoxy ring, both of which simultaneously cause ethylene oxide to be easy to open the ring and react with carbon dioxide into ethylene carbonate.

Optionally, the substituents in Formula I, Formula II and Formula III are each independently selected from any one of a hydroxyl group, an amino group, a nitro group, an aldehyde group, an ester group, a carboxyl group, a nitroso group, an amide group or a carbonyl group.

Optionally, a mass ratio of the ionic liquid to ethylene carbonate is 1:(1-10), for example, may be 1:1, 1:2, 1:3, 1:4, 1:5, 1:6, 1:7, 1:8, 1:9, 1:10 or the like.

In the present application, optionally, the mass ratio of the ionic liquid to ethylene carbonate is controlled to be within the above range. When the content of the ionic liquid is too high, the composite absorbent has increased viscosity and cannot be in full contact with ethylene oxide, which might decrease absorption efficiency of ethylene oxide. When the content of the ionic liquid is too low, the selective absorbency of the composite absorbent decreases, which affects the absorption effect of ethylene oxide. Moreover, too low a content of the ionic liquid will significantly reduce catalytic efficiency of a cycloaddition reaction of ethylene oxide and carbon dioxide at an absorption pre-reaction stage.

In a fourth aspect, the present application further provides a method for coupled absorption and conversion of ethylene oxide and co-production of ethylene carbonate. The method uses the composite absorbent as described in the first aspect or the third aspect.

The present application uses a composite of an ionic liquid and ethylene carbonate as an absorbent for the coupled absorption and conversion of ethylene oxide to achieve one-step absorption and conversion of ethylene oxide, effectively reducing energy consumption and a cost of a device.

Optionally, the method includes the following steps: making the composite absorbent in contact with a feed gas containing ethylene oxide and feeding an obtained ethylene oxide-rich absorption liquid into a main reactor to obtain ethylene carbonate.

Components of the feed gas in the present application and molar percentages thereof are 2.6% ethylene oxide, 52.77% methane, 32.53% ethylene, 4.9% oxygen, 1.5% carbon dioxide, 4.18% argon, 1% nitrogen and 0.52% ethane, respectively. The feed gas in the present application is a gas mixture after ethylene oxidation. The feed gas includes both ethylene oxide and carbon dioxide. In an absorption process, the ionic liquid can not only effectively absorb ethylene oxide and carbon dioxide, but also catalyze a reaction of ethylene oxide with carbon dioxide to produce ethylene carbonate. The absorbent implement a pre-reaction for preparing ethylene carbonate while absorbing, which reduces energy consumption and the content of ethylene oxide in a tail gas of a main reaction unit for carbonylation in a subsequent stage, ensures safety and has a good industrial application value.

In the present application, a conversion of ethylene oxide in the pre-reaction is 2-35%, for example, may be, 2%, 5%, 8%, 10%, 12%, 15%, 20%, 23%, 27%, 30%, 35% or the like.

In the present application, the conversion rate of ethylene oxide in the pre-reaction stage is only 2-35%. This is because excessive carbon dioxide cannot be provided in the pre-reaction stage and the temperature of the pre-reaction cannot support the catalyst to achieve an optimum conversion, resulting in a relatively low conversion rate of ethylene oxide in the pre-reaction stage.

Optionally, the method includes the following steps: making the composite absorbent in full countercurrent contact with a feed gas containing ethylene oxide in an absorption tower, returning a lean gas mixture with ethylene oxide removed at a top of the tower to an ethylene oxidation stage after the lean gas mixture is treated, feeding an ethylene oxide-rich absorption liquid at a bottom of the tower into a main reactor after heat exchange for a reaction to obtain a reaction liquid, recycling one part of the obtained reaction liquid containing an ionic liquid as an absorption liquid, and treating the other part of the obtained reaction liquid to obtain high-purity ethylene carbonate.

It is to be noted that in the reaction stage, carbon dioxide is added to the main reactor so that an amount of carbon dioxide is sufficient with respect to ethylene oxide, that is, sufficient carbon dioxide and the absorbed ethylene oxide are reacted into ethylene carbonate.

In the main reactor of the present application, the ionic liquid catalyzes the reaction of the absorbed ethylene oxide with carbon dioxide to produce ethylene carbonate. A current process for treating a tail gas after ethylene oxidation is to absorb ethylene oxide in an absorption tower and feeding ethylene oxide into a reactor after steam stripping and refinement to produce ethylene carbonate. However, the present application uses the composite absorbent of ethylene carbonate and the ionic liquid for coupled absorption and conversion and co-production of ethylene carbonate. The composite absorbent has relatively high absorbency for ethylene oxide, the ionic liquid has excellent performance such as low vapor pressure and low specific heat capacity. After the reaction is complete, the product can be recycled as the absorbent into the absorption tower for reuse, thereby simplifying separation and recycling processes. For a process flow, the method of the present application directly couples absorption and conversion to achieve one-step production of ethylene carbonate, which not only reduces the workload and an equipment cost, also reduces the steam stripping and the energy consumption, and satisfies the requirements for the economy, high efficiency, energy saving, and environmental protection.

Optionally, a mass ratio (liquid-gas mass ratio) of the composite absorbent to the feed gas containing ethylene oxide is (1-5):1, for example, may be 1:1, 1.5:1, 2:1, 2.3:1, 3:1, 3.5:1, 4:1, 5:1 or the like. Optionally, the mass ratio is (2-3):1.

Optionally, an operation pressure of the absorption tower is 1-5 MPa, for example, may be 1 MPa, 1.5 MPa, 2 MPa, 2.3 MPa, 3 MPa, 3.5 MPa, 4 MPa, 4.6 MPa, 5 MPa or the like.

Optionally, the operation pressure is 1.5-2.5 MPa.

Optionally, an operation temperature of the absorption tower is 50-100° C., for example, may be 50° C., 60° C., 70° C., 80° C., 90° C., 100° C. or the like. Optionally, the operation temperature is 60-80° C.

Optionally, a reaction pressure of the main reactor is 1-5 MPa, for example, may be 1 MPa, 1.2 MPa, 2 MPa, 2.5 MPa, 3 MPa, 3.4 MPa, 4 MPa, 4.5 MPa, 5 MPa or the like. Optionally, the reaction pressure is 2-3 MPa.

Optionally, a reaction temperature of the main reactor is 80-300° C., for example, may be 80° C., 90° C., 100° C., 110° C., 120° C., 130° C., 140° C., 150° C., 160° C., 170° C., 180° C., 190° C., 200° C., 210° C., 220° C., 230° C., 240° C., 250° C., 260° C., 270° C., 280° C., 290° C., 300° C. or the like. Optionally, the reaction temperature is 100-200° C.

Optionally, the reaction is conducted for 0.5-5 h, for example, may be 0.5 h, 1 h, 1.5 h, 2 h, 2.5 h, 3 h, 3.5 h, 4 h, 4.5 h, 5 h or the like. Optionally, the reaction is conducted for 1-3 h.

Optionally, the ethylene oxide-rich absorption liquid is fed into the main reactor after heat exchange to 100-200° C., which, for example, may be 100° C., 110° C., 120° C., 130° C., 140° C., 150° C., 160° C., 170° C., 180° C., 190° C., 200° C. or the like.

Optionally, one part of the reaction liquid is cooled to 60-80° C. before entering the absorption tower, which, for example, may be 60° C., 62° C., 65° C., 68° C., 70° C., 73° C., 76° C., 80° C. or the like.

Compared with the prior art, the present application has the beneficial effects below.

(1) In the present application, the composite absorbent of the ionic liquid and ethylene carbonate is used for absorbing ethylene oxide and carbon dioxide. In the absorption process, the ionic liquid may also be used as the catalyst to react the absorbed ethylene oxide and carbon dioxide into ethylene carbonate, which improves the absorption effect of ethylene oxide, reduces the desorption and refinement of ethylene oxide, and reduces the energy consumption through simultaneous pre-conversion and absorption, thus having the good industrial application value.

(2) The present application uses for the first time the composite absorbent of ethylene carbonate and the ionic liquid for the coupled absorption and conversion and the co-production of ethylene carbonate. The composite absorbent has the relatively high absorbency for ethylene oxide, the ionic liquid has the excellent performance such as the low vapor pressure and the low specific heat capacity. After the reaction is complete, the product can be recycled as the absorbent into the absorption tower for reuse, thereby simplifying the separation and recycling processes. For the process flow, the method of the present application directly couples the absorption and conversion to achieve the one-step production of ethylene carbonate, which not only requires a small amount of workload and the low equipment cost, but also reduces the steam stripping, reduces the energy consumption, and satisfies the requirements for the economy, high efficiency, energy saving, and environmental protection.

(3) In the present application, in the process of using the composite absorbent for the coupled absorption and conversion of ethylene oxide and the co-production of ethylene carbonate, the absorption rate of ethylene oxide can reach more than or equal to 98.5% in the absorption stage, and the conversion rate of ethylene oxide can reach up to 99.2% and the selectivity can reach more than or equal to 92% in the main reaction stage.

(4) The present application provides the composite absorbent containing the ionic liquid and ethylene carbonate, which can increase the selectivity for absorption and separation of ethylene oxide, effectively reduce the vapor pressure of the composite absorbent, and reduce the loss of the solvent in the desorption process. The composite absorbent has the characteristics of a simple process flow, high operation elasticity, low energy consumption, and a remarkable absorption effect and has the good industrial application prospect.

(5) The present application uses the preceding composite absorbent for separation and purification of ethylene oxide. A removal rate of ethylene oxide can reach 88.8% to 99.8% so that the absorbency of ethylene oxide in the feed gas is effectively improved. Meanwhile, the purity of ethylene oxide obtained at the top of the desorption tower is more than or equal to 80.0% so that the selective absorbency of the composite absorbent for ethylene oxide is effectively improved. The use of the composite absorbent for the separation and purification of ethylene oxide in the present application simplifies the process flow, increase the operation flexibility of the device, reduces the energy consumption, and provides technical support for the continuous production and purification of ethylene oxide.

### BRIEF DESCRIPTION OF DRAWINGS

FIG. 1 is a flowchart of separation and purification of ethylene oxide according to the present application.

FIG. 2 is a flowchart of coupled absorption and conversion of ethylene oxide and co-production of ethylene carbonate according to the present application.

### REFERENCE LIST

T1 absorption tower for ethylene oxide
T2 desorption tower for ethylene oxide
E1 heat exchanger for ethylene oxide-rich and -lean liquids
E2 cooler for an ethylene oxide-lean liquid
E3 heater for an ethylene oxide-rich liquid
1 feed gas containing ethylene oxide
2 composite absorbent
3 ethylene oxide-rich absorption liquid
4 ethylene oxide-rich absorption liquid after heat exchange
5 lean desorption liquid
6 lean desorption liquid after heat exchange
7 ethylene oxide gas after desorption
8 lean gas mixture with ethylene oxide removed
T3 absorption tower for ethylene oxide
R1 ethylene oxide reactor
a gas mixture with ethylene oxide removed
b composite absorbent
c feed gas containing ethylene oxide
d recycled CO₂ gas
e ethylene oxide-rich absorption liquid
f fresh CO₂ gas
g ethylene carbonate

### DETAILED DESCRIPTION

Technical solutions of the present application are further described below through the detailed description. Those skilled in the art are to understand that examples described herein are merely used for a better understanding of the present application and are not to be construed as specific limitations to the present application.

### Example a1

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 80° C. was fed into an absorption tower from the bottom of the tower. The composite absorbent, an ionic liquid 1-(2-hydroxyethyl)-3-methylimidazolium bromide ([Hemim]Br) and ethylene carbonate mixed uniformly at a mass ratio of 1:10, entered from the top of the absorption tower (T1). The feed gas was in countercurrent contact with the absorbent at an absorption temperature of 80° C., under an operation pressure of 2 MPa, and at a liquid-gas mass ratio of 3. A conversion rate of ethylene oxide in an absorption and pre-conversion stage was 28.8%, ethylene oxide at the top of the absorption tower had a concentration of 69 ppm, and an absorption rate of ethylene oxide was 99.9%.

The preceding absorption liquid was added to a reactor and reacted for 1 h at a reaction temperature of 125° C. and under a pressure of 2 MPa. It was obtained through detection that a conversion rate of the remaining ethylene oxide was 95.2% and the selectivity was 99.8%.

### Example a2

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 80° C. was fed into an absorption tower from the bottom of the tower. The composite absorbent, an ionic liquid 1-hexyl-3-methylimidazolium bromide ([Hmim]Br) and ethylene carbonate mixed uniformly at a mass ratio of 1:10, entered from the top of the absorption tower (T1). The feed gas was in countercurrent contact with the absorbent at an absorption temperature of 80° C., under an operation pressure of 2 MPa, and at a liquid-gas mass ratio of 3. A conversion rate of ethylene oxide in an absorption and pre-conversion stage was 15.8%, ethylene oxide at the top of the absorption tower had a concentration of 227 ppm, and an absorption rate of ethylene oxide was 99.5%.

The preceding absorption liquid was added to a reactor and reacted for 3 h at a reaction temperature of 120° C. and under a pressure of 2 MPa. It was obtained through detection that a conversion rate of the remaining ethylene oxide was 52.5% and the selectivity was 99.8%.

### Example a3

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 80° C. was fed into an absorption tower from the bottom of the tower. The composite absorbent, an ionic liquid 1-(2-hydroxyethyl)-3-methylimidazolium bromide ([Hemim]Br) and ethylene carbonate mixed uniformly at a mass ratio of 1:9, entered from the top of the absorption tower (T1). The feed gas was in countercurrent contact with the absorbent at an absorption temperature of 80° C., under an operation pressure of 2 MPa, and at a liquid-gas mass ratio of 3. A conversion rate of ethylene oxide in an absorption and pre-conversion stage was 32.2%, ethylene oxide at the top of the absorption tower had a concentration of 44 ppm, and an absorption rate of ethylene oxide was 99.9%.

The preceding absorption liquid was added to a reactor and reacted for 1 h at a reaction temperature of 120° C. and under a pressure of 2 MPa. It was obtained through detection that a conversion rate of the remaining ethylene oxide was 99.2% and the selectivity was 99.8%.

### Example a4

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 80° C. was fed into an absorption tower from the bottom of the tower. The composite absorbent, an ionic liquid 2-hydroxyethyl tributyl ammonium bromide (HETBAB) and ethylene carbonate mixed uniformly at a mass ratio of 1:10, entered from the top of the absorption tower (T1). The feed gas was in countercurrent contact with the absorbent at an absorption temperature of 80° C., under an operation pressure of 2 MPa, and at a liquid-gas mass ratio of 3. A conversion rate of ethylene oxide in an absorption and pre-conversion stage was 29.2%, ethylene oxide at the top of the absorption tower had a concentration of 55 ppm, and an absorption rate of ethylene oxide was 99.9%.

The preceding absorption liquid was added to a reactor and reacted for 1 h at a reaction temperature of 125° C. and under a pressure of 2 MPa. It was obtained through detection that a conversion of the remaining ethylene oxide was 95.8% and the selectivity was 99.8%.

### Example a5

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 80° C. was fed into an absorption tower from the bottom of the tower. The composite absorbent, an ionic liquid ethylidene bis(triphenylphosphonium bromide) (E[TPB]) and ethylene carbonate mixed uniformly at a mass ratio of 1:10, entered from the top of the absorption tower (T1). The feed gas was in countercurrent contact with the absorbent at an absorption temperature of 80° C., under an operation pressure of 3 MPa, and at a liquid-gas mass ratio of 3. A conversion rate of ethylene oxide in an absorption and pre-conversion stage was 25.6%, ethylene oxide at the top of the absorption tower had a concentration of 95 ppm, and an absorption rate of ethylene oxide was 99.8%.

The preceding absorption liquid was added to a reactor and reacted for 3 h at a reaction temperature of 160° C. and under a pressure of 3 MPa. It was obtained through detection that a conversion of the remaining ethylene oxide was 92.9% and the selectivity was 99%.

### Example a6

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 60° C. was fed into an absorption tower from the bottom of the tower. The composite absorbent, an ionic liquid 1-(2-hydroxyethyl)-3-methylimidazolium bromide ([Hemim]Br) and ethylene carbonate mixed uniformly at a mass ratio of 1:1, entered from the top of the absorption tower (T1). The feed gas was in countercurrent contact with the absorbent at an absorption temperature of 60° C., under an operation pressure of 2 MPa, and at a liquid-gas mass ratio of 2. A conversion rate of ethylene oxide in an absorption and pre-conversion stage was 18.2%, ethylene oxide at the top of the absorption tower 2 had a concentration of 152 ppm, and an absorption rate of ethylene oxide was 99.4%.

The preceding absorption liquid was added to a reactor and reacted for 0.5 h at a reaction temperature of 80° C. and under a pressure of 5 MPa. It was obtained through detection that a conversion of the remaining ethylene oxide was 65.5% and the selectivity was 98.2%.

### Example a7

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 100° C. was fed into an absorption tower from the bottom of the tower. The composite absorbent, an ionic liquid 1-(2-hydroxyethyl)-3-methylimidazolium bromide ([Hemim]Br) and ethylene carbonate mixed uniformly at a mass ratio of 1:5, entered from the top of the absorption tower (T1). The feed gas was in countercurrent contact with the absorbent at an absorption temperature of 100° C., under an operation pressure of 2 MPa, and at a liquid-gas mass ratio of 3. A conversion rate of ethylene oxide in an absorption and pre-conversion stage was 30.1%, ethylene oxide at the top of the absorption tower had a concentration of 395 ppm, and an absorption rate of ethylene oxide was 98.5%.

The preceding absorption liquid was added to a reactor and reacted for 5 h at a reaction temperature of 300° C. and under a pressure of 2 MPa. It was obtained through detection that a conversion of the remaining ethylene oxide was 95.2% and the selectivity was 98.2%.

### Example a8

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 60° C. was fed into an absorption tower from the bottom of the tower. The composite absorbent, an ionic liquid 2-hydroxyethyl tributyl ammonium bromide (HETBAB) and ethylene carbonate mixed uniformly at a mass ratio of 1:7, entered from the top of the absorption tower (T1). The feed gas was in countercurrent contact with the absorbent at an absorption temperature of 60° C., under an operation pressure of 2 MPa, and at a liquid-gas mass ratio of 2. A conversion rate of ethylene oxide in an absorption and pre-conversion stage was 17.2%, ethylene oxide at the top of the absorption tower had a concentration of 108 ppm, and an absorption rate of ethylene oxide was 99.5%.

The preceding absorption liquid was added to a reactor and reacted for 2 h at a reaction temperature of 200° C. and under a pressure of 1.5 MPa. It was obtained through detection that a conversion of the remaining ethylene oxide was 85.1% and the selectivity was 92%.

### Example a9

This example provides a composite absorbent and a method thereof for coupled absorption and conversion of ethylene oxide and co-production of ethylene carbonate. This example differs from Example a1 only in that a mass ratio of 1-(2-hydroxyethyl)-3-methylimidazolium bromide ([Hemim]Br) and ethylene carbonate was 1:12.

A conversion of ethylene oxide in an absorption and pre-conversion stage was 17%, ethylene oxide at the top of the absorption tower had a concentration of 158 ppm, and an absorption rate of ethylene oxide was 99.4%. The preceding absorption liquid was added to a reactor. After the reaction, it was obtained through detection that a conversion of the remaining ethylene oxide was 95.2% and the selectivity was 99.8%.

### Comparative Example a1

This example provides a composite absorbent and a method thereof for coupled absorption and conversion of ethylene oxide and co-production of ethylene carbonate. Specific operation steps are described below.

A feed gas containing 2.26 mol % of ethylene oxide at a temperature of 80° C. was fed into an absorption tower from the bottom of the tower. The absorbent, ethylene carbonate, entered from the top of the absorption tower (T1). The feed gas was in countercurrent contact with the absorbent at an absorption temperature of 80° C., under an operation pressure of 2 MPa, and at a liquid-gas mass ratio of 3. Ethylene oxide at the top of the absorption tower had a concentration of 450 ppm, and an absorption rate of ethylene oxide was 98.2%.

The preceding absorption liquid was added to a main reactor, added with a catalyst 1-butyl-3-methylimidazolium bromide ([bmim]Br), and reacted for 3 h at a reaction temperature of 120° C. and under a pressure of 2 MPa. It was obtained through detection that a conversion rate of the remaining ethylene oxide was 51.6% and the selectivity was 99.9%.

As can be seen from examples and detection results, in the process of using the composite absorbent of the present application in coupled absorption and conversion of ethylene oxide for co-production of ethylene carbonate, the absorption rate of ethylene oxide can reach more than or equal to 98.5% in the absorption stage, which indicates that the composite absorbent of the ionic liquid and ethylene carbonate has relatively high absorbency for ethylene oxide. Meanwhile, the ionic liquid has excellent performance such as low vapor pressure and low specific heat capacity so that after the reaction is complete, the product can be recycled as the absorbent into the absorption tower for reuse, which simplifies separation and recycling processes. The conversion rate of ethylene oxide is 15.8-32.2% in the absorption stage and can reach up to 99.2% in the main reaction stage, and the selectivity are both more than or equal to 99%. For the whole process flow, the present application directly couples absorption and conversion of ethylene oxide to achieve one-step production of ethylene carbonate, which not only reduces the workload and an equipment cost, also reduces steam stripping and energy consumption, and satisfies the requirements for economy, high efficiency, energy saving and environmental protection.

Compared to Example a1, Comparative Example a1 uses only ethylene carbonate in the absorption stage of ethylene oxide and then implements the esterification of ethylene oxide and carbon dioxide by adding the catalyst in the main reaction stage. As can be seen from data, in the absorption stage, the absorption rate of ethylene oxide in Comparative Example a1 is lower than the absorption efficiency of ethylene oxide when the composite absorbent containing the ionic liquid is used, and in the main reaction stage, the conversion rate of ethylene oxide is lower than the conversion rate of ethylene oxide in Example a1 using the ionic liquid to catalyze the main reaction. This indicates that the addition of the ionic liquid during the absorption and conversion of ethylene oxide can not only enhances the selective absorbency for ethylene oxide but also improve catalytic efficiency for the esterification of ethylene oxide and carbon dioxide.

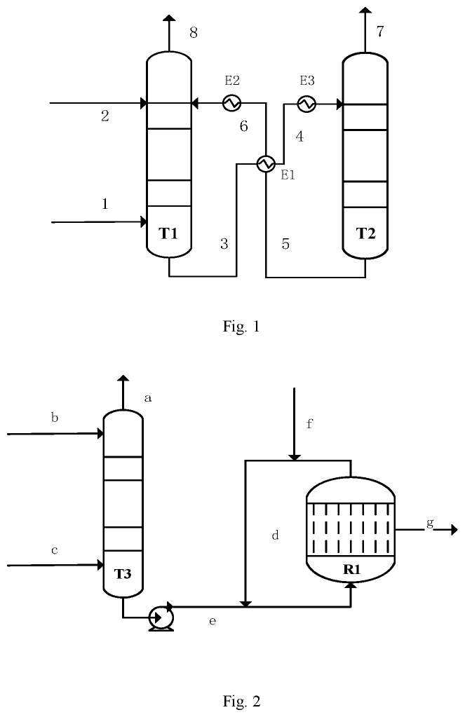

A process flow for separation and purification of ethylene oxide in the present application is shown in FIG. 1 and specifically includes the steps below. A gas mixture 1 containing ethylene oxide enters an absorption tower T1 from the bottom of the tower column. A composite absorbent 2 enters from the top of the absorption tower T1. The content of ethylene oxide 8 at the top of the absorption tower is measured after absorption. An ethylene oxide-containing composite absorbent 3 is heated to a certain temperature through heat exchange to obtain an ethylene oxide-containing absorbent 4 having a higher temperature. The ethylene oxide-containing absorbent 4 enters a desorption tower T2 and desorbed to obtain ethylene oxide 7 at the top of the desorption tower T2. A composite absorbent 5 with gas separated is subjected to heat exchange to obtain a composite absorbent 6 having a certain temperature. The composite absorbent 6 enters the absorption tower T1 to be recycled as the absorbent.

### Example b1

This group of examples provides a composite absorbent and a method thereof for separation and purification of ethylene oxide. The composite absorbent consists of an ionic liquid 1-hydroxyethyl-3-methylimidazolium hexafluorophosphate ([Hemim][PF₆]) and ethylene carbonate. The composite absorbent is used for absorption and desorption of a feed gas containing ethylene oxide. Specific operations are described below.

A gas mixture containing 2.26% of ethylene oxide at a temperature of 50° C. was fed into the absorption tower T1 from the bottom of the tower. The ionic liquid [Hemim][PF₆] and ethylene carbonate were mixed uniformly and entered from the top of the absorption tower T1. The content of ethylene oxide at the top of the tower was measured after absorption. The ethylene oxide-containing absorbent was heated to a certain temperature through heat exchange and entered the desorption tower T2 having a pressure of 50 kPa. The content of ethylene oxide at the top of the tower was obtained after desorption. The degassed ionic liquid and ethylene carbonate were subjected to heat exchange to 50° C. and then entered the absorption tower T1 to be recycled as the absorbent. Specific parameters and corresponding results are shown in Table 1.

TABLE 1

| | Temper- | | | | | | | |
|---|---|---|---|---|---|---|---|---|
| | ature of | | | | | | | |
| | the | | Pressure of | | EO at the | | | |
| | Absorp- | Content | the | Liquid- | Top of the | | EO at the | |
| | tion | of the | Absorption | Gas | Absorption | Desorption | Top of the | Removal |
| | Tower | Ionic | Tower | Molar | Tower | Temper- | Desorption | Rate of EO |
| | (° C.) | Liquid | (MPa) | Ratio | (ppm) | ature (° C.) | Tower (%) | (%) |
| Example | 50 | 30% | 2 | 2 | 276 | 130 | 97.1 | 98.9 |
| b1-1 | | | | | | | | |
| Example | 50 | 40% | 2 | 2 | 625 | 130 | 97.4 | 97.6 |
| b1-2 | | | | | | | | |
| Example | 50 | 50% | 2 | 2 | 1007 | 130 | 97.8 | 96.1 |
| b1-3 | | | | | | | | |
| Example | 50 | 60% | 2 | 2 | 1319 | 130 | 98.2 | 94.9 |
| b1-4 | | | | | | | | |
| Example | 50 | 20% | 2 | 2 | 212 | 130 | 95.3 | 99.3 |
| b1-5 | | | | | | | | |
| Example | 60 | 40% | 2 | 2.5 | 657 | 100 | 96 | 97.5 |
| b1-6 | | | | | | | | |

As can be seen from the data in Table 1, when the composite absorbent of 1-hydroxyethyl-3-methylimidazolium hexafluorophosphate and ethylene carbonate is used for the separation and purification of ethylene oxide, the removal rate of ethylene oxide can reach 94.9% to 99.3%, which indicates that the addition of the ionic liquid can improve an ability to absorb and separate ethylene oxide and achieve a significant absorption effect. In addition, it can be seen that the purity of ethylene oxide at the top of the desorption tower obtained through separation and purification of ethylene oxide using the composite absorbent described above is all higher than 95.3%, that is, the selective absorbency for ethylene oxide in the feed gas is significantly improved.

As can be seen from the comparison of Examples b1-1, b1-2, b1-3, b1-4 and b1-5, the purity of EO at the top of the desorption tower shows the tendency to significantly increase as the content of the ionic liquid increases. This indicates that the higher the content of the ionic liquid, the higher the selective absorbency for ethylene oxide in the feed gas.

As can be seen from the comparison of Examples b1-1, b1-2, b1-3 and b1-4, the removal rate of EO shows the tendency to decrease as the content of the ionic liquid increases. This is because the ionic liquid has large viscosity. A relatively high content of the ionic liquid affects the absorption effect of ethylene oxide. However, as can be seen from the data of Example b1-5, too low a content of the ionic liquid will significantly reduce the concentration of EO at the top of the desorption tower, that is, reduce the selective absorbency of the composite absorbent for ethylene oxide.

### Example b2

This group of examples provides a composite absorbent and a method thereof for separation and purification of ethylene oxide. The composite absorbent consists of an ionic liquid 1-hydroxyethyl-3-methylimidazolium tetrafluoroborate ([Hemim][BF₄]) and ethylene carbonate. The composite absorbent is used for absorption and desorption of a feed gas containing ethylene oxide. Specific operations are described below.

A gas mixture containing 2.26% of ethylene oxide at a temperature of 60° C. was fed into the absorption tower T1 from the bottom of the tower. The ionic liquid [Hemim][BF₄] and ethylene carbonate were mixed uniformly and entered from the top of the absorption tower T1. The content of ethylene oxide at the top of the tower was measured after absorption. The ethylene oxide-containing absorbent was heated to a certain temperature through heat exchange and entered the desorption tower T2 having a pressure of 60 kPa. The content of ethylene oxide at the top of the tower was obtained after desorption. The degassed ionic liquid and ethylene carbonate were subjected to heat exchange to 60° C. and then entered the absorption tower T1 to be recycled as the absorbent. Specific parameters and corresponding results are shown in Table 2.

TABLE 2

| | Temper- | | | | | | | |
|---|---|---|---|---|---|---|---|---|
| | ature of | | | | | | | |
| | the | | Pressure of | | EO at the | | | |
| | Absorp- | Content | the | | Top of the | | EO at the | |
| | tion | of the | Absorption | Liquid- | Absorption | Desorption | Top of the | Removal |
| | Tower | Ionic | Tower | Gas | Tower | Temper- | Desorption | Rate of EO |
| | (° C.) | Liquid | (MPa) | Ratio | (ppm) | ature (° C.) | Tower (%) | (%) |
| Example | 50 | 30% | 2 | 2 | 117 | 130 | 82.4 | 99.6 |
| b2-1 | | | | | | | | |
| Example | 50 | 40% | 2 | 2 | 327 | 130 | 83.5 | 98.7 |
| b2-2 | | | | | | | | |
| Example | 50 | 50% | 2 | 2 | 620 | 130 | 85.1 | 97.6 |
| b2-3 | | | | | | | | |
| Example | 50 | 60% | 2 | 2 | 887 | 130 | 86.1 | 96.6 |
| b2-4 | | | | | | | | |
| Example | 60 | 40% | 2 | 2.5 | 271 | 100 | 83.2 | 98.9 |
| b2-5 | | | | | | | | |
| Example | 80 | 50% | 2.5 | 3 | 1791 | 130 | 83.7 | 93.1 |
| b2-6 | | | | | | | | |

As can be seen from the data in Table 2, when the composite absorbent of 1-hydroxyethyl-3-methylimidazolium tetrafluoroborate and ethylene carbonate is used for the separation and purification of ethylene oxide, the removal rate of ethylene oxide can reach 93.1% to 99.6%, which indicates that the addition of the ionic liquid can improve the ability to absorb and separate ethylene oxide and achieve the significant absorption effect. Meanwhile, the purity of ethylene oxide at the top of the desorption tower and obtained through separation and purification of ethylene oxide using the composite absorbent described above is higher than 82.4%, that is, the selective absorbency for ethylene oxide in the feed gas is significantly improved.

As can be seen from the comparison of Example b2-1, b2-2, b2-3 and b2-4, the removal rate of EO shows the tendency to decrease while the concentration of EO at the top of the desorption tower shows the tendency to increase as the content of the ionic liquid increases. The same conclusions are obtained based on the data in Table 2 as the data in Table 1.

### Example b3

This group of examples provides a composite absorbent and a method thereof for separation and purification of ethylene oxide. The composite absorbent consists of an ionic liquid 1-aminoethyl-3-methylimidazolium tetrafluoroborate ([C₂NH₂mim][BF₄]) and ethylene carbonate. The composite absorbent is used for absorption and desorption of a feed gas containing ethylene oxide. Specific operations are described below.

A gas mixture containing 2.26% ethylene oxide at a temperature of 70° C. was fed into the absorption tower T1 from the bottom of the tower. The ionic liquid [C₂NH₂mim][BF₄] and ethylene carbonate were mixed uniformly and entered from the top of the absorption tower T1. The content of ethylene oxide at the top of the tower was measured after absorption. The ethylene oxide-containing absorbent was heated to a certain temperature through heat exchange and entered the desorption tower T2 having a pressure of 70 kPa. The content of ethylene oxide at the top of the tower was obtained after desorption. The degassed ionic liquid and ethylene carbonate were subjected to heat exchange to 70° C. and then entered the absorption tower T1 to be recycled as the absorbent. Specific parameters and corresponding results are shown in Table 3.

TABLE 3

| | Temper- | | | | | | | |
|---|---|---|---|---|---|---|---|---|
| | ature of | | | | | | | |
| | the | | Pressure of | | EO at the | | | |
| | Absorp- | Content | the | | Top of the | | EO at the | |
| | tion | of the | Absorption | Liquid- | Absorption | Desorption | Top of the | Removal |
| | Tower | Ionic | Tower | Gas | Tower | Temper- | Desorption | Rate of EO |
| | (° C.) | Liquid | (MPa) | Ratio | (ppm) | ature (° C.) | Tower (%) | (%) |
| Example | 50 | 30% | 2 | 2 | 66 | 130 | 80.6 | 99.8 |
| b3-1 | | | | | | | | |
| Example | 50 | 40% | 2 | 2 | 236 | 130 | 81.5 | 99.1 |
| b3-2 | | | | | | | | |
| Example | 50 | 50% | 2 | 2 | 470 | 130 | 82.4 | 98.2 |
| b3-3 | | | | | | | | |
| Example | 50 | 60% | 2 | 2 | 717 | 130 | 83.2 | 97.2 |
| b3-4 | | | | | | | | |
| Example | 60 | 40% | 2 | 2.5 | 146 | 100 | 82.6 | 99.4 |
| b3-5 | | | | | | | | |
| Example | 80 | 50% | 2.5 | 3 | 1370 | 130 | 82.8 | 94.7 |
| b3-6 | | | | | | | | |

As can be seen from the data in Table 3, when the composite absorbent of 1-aminoethyl-3-methylimidazolium tetrafluoroborate and ethylene carbonate is used for the separation and purification of ethylene oxide, the removal rate of ethylene oxide can reach 94.7% to 99.8%, which indicates that the addition of the ionic liquid can improve the ability to absorb and separate ethylene oxide and achieve the significant absorption effect.

As can be seen from the comparison of Example b3-1, b3-2, b3-3 and b3-4, as the content of the ionic liquid increases, the removal rate of EO shows the tendency to decrease, while the concentration of EO at the top of the desorption tower shows the tendency to increase, that is, the selectivity of EO shows the tendency to increase. The same conclusions are obtained based on the data in Table 3 as the data in Table 1 and Table 2.

The ionic liquid 1-hydroxyethyl-3-methylimidazolium tetrafluoroborate is used in Example b2 and the ionic liquid 1-aminoethyl-3-methylimidazolium tetrafluoroborate is used in Example b3. These two ionic liquids differ only in that cations have different substituents. The substituent in the cation of the ionic liquid in Example b2 is a hydroxyl group, and the substituent in the cation of the ionic liquid in Example b3 is an amino group. As can be seen from the comparison of the data in Table 2 and Table 3, when the substituent in the cation is a hydroxyl group, ethylene oxide at the top of the desorption tower has a higher purity and a lower removal rate, that is, the selectivity of the hydroxyl group for absorption and separation of ethylene oxide is greater than that of the amino group, but the absorbency of the hydroxyl group is lower than that of the amino group.

### Comparative Example b1

This group of examples provides an absorbent and a method thereof for separation and purification of ethylene oxide. These examples differ from Example b1 only in that the absorbent consists of only ethylene carbonate.

Specific parameters and corresponding results are shown in Table 4.

TABLE 4

| | Temper- | | | | | | |
|---|---|---|---|---|---|---|---|
| | ature of | | | | | | |
| | the | Pressure of | | EO at the | | | |
| | Absorp- | the | | Top of the | | EO at the | |
| | tion | Absorption | Liquid- | Absorption | Desorption | Top of the | Removal |
| | Tower | Tower | Gas | Tower | Temper- | Desorption | Rate of EO |
| | (° C.) | (MPa) | Ratio | (ppm) | ature (° C.) | Tower (%) | (%) |
| Comparative | 50 | 2 | 2 | 86 | 130 | 74 | 99.6 |
| Example b1-1 | | | | | | | |
| Comparative | 60 | 2 | 2.5 | 241 | 100 | 80.1 | 99 |
| Example b1-2 | | | | | | | |
| Comparative | 50 | 2.5 | 2 | 531 | 130 | 77.8 | 98 |
| Example b1-3 | | | | | | | |
| Comparative | 80 | 2.5 | 3 | 1230 | 130 | 79.9 | 95.3 |
| Example b1-4 | | | | | | | |

As can be seen from the comparison of the data in Table 4 with the data in Tables 1 to 3, the purity of ethylene oxide at the top of the desorption tower when the absorbent containing only ethylene carbonate is used for separation and purification of ethylene oxide is significantly lower than that when the composite absorbent containing the ionic liquid is used. This indicates that the selectivity of single ethylene carbonate for ethylene oxide is significantly lower than that of the composite absorbent of the ionic liquid and ethylene carbonate, that is, the addition of the ionic liquid can improve the selectivity for absorption and separation of ethylene oxide and achieve a significant absorption effect.

### Comparative Example b2

This comparative example provides a composite absorbent and a method thereof for separation and purification of ethylene oxide. This comparative example differs from Example b1 only in that 1-hydroxyethyl-3-methylimidazolium hexafluorophosphate ([Hemim][PF₆]) was replaced with water. Specific operations are described below.

A gas mixture containing 2.26% of ethylene oxide at a temperature of 50° C. was fed into the absorption tower T1 from the bottom of the tower. Water and ethylene carbonate were mixed uniformly and entered from the top of the absorption tower T1. The content of ethylene oxide at the top of the tower was measured after absorption. The ethylene oxide-containing absorbent was heated to a certain temperature through heat exchange and entered the desorption tower T2 having a pressure of 50 kPa. The content of ethylene oxide at the top of the tower was obtained after desorption. The degassed water and ethylene carbonate were subjected to heat exchange to 50° C. and then entered the absorption tower T1 to be recycled as the absorbent. Specific parameters and corresponding results are shown in Table 5.

Specific parameters and corresponding results are shown in Table 5.

TABLE 5

| | Temper- | | | | | | | |
|---|---|---|---|---|---|---|---|---|
| | ature of | | | | | | | |
| | the | | Pressure of | | EO at the | | | |
| | Absorp- | Content | the | | Top of the | | EO at the | |
| | tion | of the | Absorption | Liquid- | Absorption | Desorption | Top of the | Removal |
| | Tower | Ionic | Tower | Gas | Tower | Temper- | Desorption | Rate of EO |
| | (° C.) | Liquid | (MPa) | Ratio | (ppm) | ature (° C.) | Tower (%) | (%) |
| Comparative | 50 | 30% | 2 | 2 | 453 | 130 | 62.3 | 98.2 |
| Example b2-1 | | | | | | | | |
| Comparative | 60 | 40% | 2 | 2.5 | 953 | 100 | 70.5 | 96.3 |
| Example b2-2 | | | | | | | | |
| Comparative | 50 | 60% | 2.5 | 2 | 1835 | 130 | 67.2 | 92.9 |
| Example b2-3 | | | | | | | | |
| Comparative | 80 | 50% | 2.5 | 3 | 3099 | 130 | 39.5 | 88.1 |
| Example b2-4 | | | | | | | | |

As can be seen from the comparison of the data in Table 5 with the data in Tables 1 to 3, the concentration of ethylene oxide obtained at the top of the desorption tower through the separation and purification of ethylene oxide using the composite absorbent containing the ionic liquid of the present application is significantly higher than that obtained by using the composite absorbent of water and ethylene carbonate, which indicates that the selective absorbency of the composite absorbent containing the ionic liquid is significantly higher than that of the composite absorbent of water and ethylene carbonate. This indicates that the composite absorbent containing the ionic liquid of the present application has a high absorbency and a significant absorption effect, which provides a new option for efficient separation and purification of ethylene oxide.

The applicant has stated that the above are only specific examples of the present application, and the scope of the present application is not limited thereto.

1. A composite absorbent, comprising an ionic liquid and ethylene carbonate, wherein the ionic liquid has a structure represented by Formula I, Formula II or Formula III: 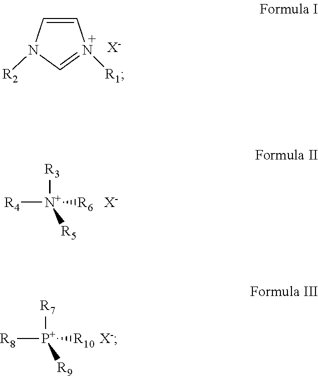
wherein R₁ and R₂ in Formula I are each independently selected from any one of substituted or unsubstituted C1-C6 alkyl, substituted or unsubstituted C2-C6 alkenyl, substituted or unsubstituted C2-C6 alkynyl, substituted or unsubstituted C6-C30 aryl, substituted or unsubstituted C3-C30 heteroaryl or substituted or unsubstituted C1-C6 alkoxy; and
wherein an anion X⁻ in Formula I is selected from any one of BF₄⁻, PF₆⁻, Tf₂N⁻, RCOO⁻, Cl⁻ or Br⁻; wherein R is selected from any one of alkyl, alkenyl or alkynyl;
wherein R₃, R₄, R₅ and R₆ in Formula II are each independently selected from any one of substituted or unsubstituted C1-C6 alkyl, substituted or unsubstituted C2-C6 alkenyl, substituted or unsubstituted C2-C6 alkynyl, substituted or unsubstituted C6-C30 aryl. substituted or unsubstituted C3-C30 heteroaryl or substituted or unsubstituted C1-C6 alkoxy:
wherein R₇, R₈, R₉ and R₁₀ in Formula III are each independently selected from any one of substituted or unsubstituted C1-C6 alkyl, substituted or unsubstituted C2-C6 alkenyl, substituted or unsubstituted C2-C6 alkynyl, substituted or unsubstituted C6-C30 aryl. substituted or unsubstituted C3-C30 heteroaryl or substituted or unsubstituted C1-C6 alkoxy; and
wherein anions X⁻ in Formula II and Formula III are each independently selected from any one of Cl⁻, Br⁻ or I⁻.

2. The composite absorbent according to claim 1, wherein the anion X⁻ is selected from any one of BF₄⁻, PF₆⁻, Tf₂N⁻ or RCOO⁻, optionally, BF₄⁻ or PF₆⁻.

3. The composite absorbent according to claim 1, wherein the substituents in R₁ and R₂ are each independently selected from any one of a hydroxyl group, an amino group, a nitro group, an aldehyde group, an ester group, a carboxyl group or a sulfhydryl group;
optionally, the substituents in R₁ and R₂ are a hydroxyl group or an amino group.

4. The composite absorbent according to claim 1, wherein the ionic liquid is selected from any one or a combination of at least two of 1-hydroxyethyl-3-methylimidazolium hexafluorophosphate, 1-hydroxyethyl-3-methylimidazolium tetrafluoroborate, 1-aminoethyl-3-methylimidazolium tetrafluoroborate, 1-aminoethyl-3-methylimidazolium hexafluorophosphate, 1-hydroxyethyl-3-ethylimidazolium hexafluorophosphate or 1-hydroxyethyl-3-ethylimidazolium tetrafluoroborate;
optionally, a mass percentage of the ionic liquid in the composite absorbent is 10-60%, optionally 30-50%.

5. A method for separation and purification of ethylene oxide, wherein the method uses the composite absorbent according to claim 2.

6. The method according to claim 5, comprising the following steps: making the composite absorbent in contact with a feed gas containing ethylene oxide, returning a lean gas mixture with ethylene oxide removed to an ethylene oxidation stage, and subjecting an ethylene oxide-rich absorption liquid to desorbing to obtain ethylene oxide.

7. The method according to claim 5, comprising the following steps: making the composite absorbent in full countercurrent contact with a feed gas containing ethylene oxide in an absorption tower, returning a lean gas mixture with ethylene oxide removed at a top of the tower to an ethylene oxidation stage after the lean gas mixture is treated, feeding an ethylene oxide-rich absorption liquid at a bottom of the tower into a desorption tower after heat exchange, followed by collecting a desorbed gas phase at a top of the tower to obtain ethylene oxide, and returning a lean desorption liquid in a desorbed liquid phase at the bottom of the tower to the absorption tower after heat exchange.

8. The method according to claim 7, wherein a molar concentration of ethylene oxide in the feed gas is 0.1-5%, optionally 2-3%.

9. The method according to claim 7, wherein the absorption tower has an operation pressure of 0.1-5 MPa, optionally 1-3 MPa;
optionally, the absorption tower has an operation temperature of 40-100° C., optionally 50-80° C.;
optionally, a molar ratio of the composite absorbent to the feed gas is (1-4):1, optionally (2-3):1;
optionally, the desorption tower has an operation pressure of 10-150 kPa, optionally 50-150 kPa;
optionally, the desorption tower has an operation temperature of 80-150° C.; optionally 90-130° C.

10. The method according to claim 7, wherein the lean desorption liquid is recycled back to the absorption tower after heat exchange to 50-80° C.

11. The method according to claim 7, wherein the ethylene oxide-rich absorption liquid is fed into the desorption tower after heat exchange to 90-130° C.

12. (canceled)

13. The composite absorbent according to claim 1, wherein the substituents in Formula I, Formula II and Formula III are each independently selected from any one of a hydroxyl group, an amino group, a nitro group, an aldehyde group, an ester group, a carboxyl group, a nitroso group, an amide group or a carbonyl group.

14. The composite absorbent according to claim 1, wherein a mass ratio of the ionic liquid to ethylene carbonate is 1:(1-10).

15. A method for coupled absorption and conversion of ethylene oxide and co-production of ethylene carbonate, wherein the method uses the composite absorbent according to claim 1.

16. The method according to claim 15, comprising the following steps: making the composite absorbent in contact with a feed gas containing ethylene oxide and feeding an obtained ethylene oxide-rich absorption liquid into a main reactor to obtain ethylene carbonate.

17. The method according to claim 16, comprising the following steps: making the composite absorbent in full countercurrent contact with a feed gas containing ethylene oxide in an absorption tower, returning a lean gas mixture with ethylene oxide removed at a top of the tower to an ethylene oxidation stage after the lean gas mixture is treated, feeding an ethylene oxide-rich absorption liquid at a bottom of the tower into a main reactor after heat exchange for a reaction to obtain a reaction liquid, recycling one part of the obtained reaction liquid containing an ionic liquid as an absorption liquid, and treating the other part of the obtained reaction liquid to obtain high-purity ethylene carbonate.

18. The method according to claim 17, wherein a mass ratio of the composite absorbent to the feed gas containing ethylene oxide is (1-5):1, optionally (2-3):1.

19. The method according to claim 17, wherein the absorption tower has an operation pressure of 1-5 MPa, optionally 1.5-2.5 MPa;
optionally, the absorption tower has an operation temperature of 50-100° C., optionally 60-80° C.;
optionally, the main reactor has a reaction pressure of 1-5 MPa, optionally 2-3 MPa;
optionally, the main reactor has a reaction temperature of 80-300° C., optionally 100-200° C.;
optionally, the reaction is conducted for 0.5-5 h, optionally 1-3 h.

20. The method according to claim 17 wherein the ethylene oxide-rich absorption liquid is fed into the main reactor after heat exchange to 100-200° C.

21. The method according to claim 17, wherein the one part of the reaction liquid is cooled to 60-80° C. before entering the absorption tower.
